(19) United States
(12) Patent Application Publication
(10) Pub. No.: US 2007/0123543 A1
(43) Pub. Date: May 31, 2007

(54) Novel compounds

(76) Inventors: Nafizal Hossain, Lund (SE); Svetlana Ivanova, Lund (SE); Marguerite Mensonides-Harsema, Lund (SE)

Correspondence Address: FISH & RICHARDSON P.C., P.O BOX 1022, MINNEAPOLIS, MN, 55440-1022, US

(21) Appl. No.: 10/581,171

(22) PCT Filed: Nov. 30, 2004
(86) PCT No.: PCT/SE04/01771

§ 371 Date: May 31, 2006

(30) Foreign Application Priority Data
Dec. 5, 2003 (SE) 0303280-2

(51) Int. Cl. A61K 31/517; A61K 31/506; C07D 491/04

(52) U.S. Cl. 514/256; 546/17; 514/278; 544/230; 514/266.21

(57) ABSTRACT

The invention provides compounds of formula (I) wherein m, R¹, n, R², q, X, Y, R³, R⁴, R⁵, R⁶, R⁷ and R⁸ are as defined in the specification, processes for their preparation, pharmaceutical compositions containing them and their use in therapy.

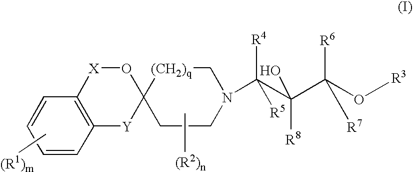

The present invention relates to novel compounds, processes for their preparation, pharmaceutical compositions containing them and their use in therapy.

Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C-X-C) and Cys-Cys (C-C) families. These are distinguished on the basis of a single amino acid insertion between the NH-proximal pair of cysteine residues and sequence similarity.

The C-X-C chemokines include several potent chemoattractants and activators of neutrophils such as interleukin-8 (IL-8) and neutrophil-activating peptide 2 (NAP-2).

The C-C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

In accordance with the present invention, there is therefore provided a compound of formula 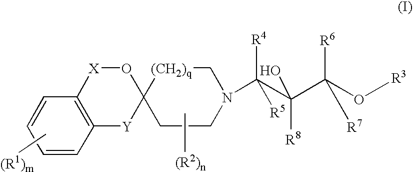
wherein
m is 0, 1, 2, 3 or 4;
each R¹ independently represents halogen, cyano, hydroxyl, C₁-C₆ alkyl, C₁-C₆ haloalkyl, C₁-C₆ alkoxy, C₁-C₆ alkylsulphonyl or sulphonamido (—SO₂NH₂);
X represents a bond or —CH₂— and Y represents a bond or —CH₂—, provided that X and Y do not both simultaneously represent a bond or —CH₂—;
n is 0, 1 or 2;
each R² independently represents halogen, C₁-C₆ alkyl or C₁-C₆ haloalkyl;
q is 0 or 1;
R³ represents a saturated or unsaturated 5- to 10-membered ring system other than phenyl, which ring system may comprise at least one ring heteroatom selected from is nitrogen, oxygen and sulphur, the ring system being optionally substituted with at least one substituent selected from halogen, cyano, oxo, nitro, hydroxyl, carboxyl, —C(O)H, —NR⁹R¹⁰, —C(O)NR¹¹R¹², —NHC(O)R¹³, —NHSO₂R¹⁴, —SO₂NR¹⁵R¹⁶, —NHC(O)NR¹⁷R¹⁸, C₁-C₆ alkyl, C₁-C₆ alkoxy, C₁-C₆ alkylthio, C₁-C₆ alkylsulphonyl, C₁-C₆ haloalkyl, C₁-C₆ alkoxyC₁-C₆ alkyl, C₁-C₆ alkylcarbonyl, phenylcarbonyl, C₃-C₆ cycloalkyl, C₃-C₆ cycloalkylmethyl and a saturated or unsaturated 5- to 6-membered heterocyclic ring comprising at least one ring heteroatom selected from nitrogen, oxygen and sulphur;
R⁴, R⁵, R⁶, R⁷ and R⁸ each independently represent hydrogen, halogen, C₁-C₆ alkyl or C₁-C₆ haloalkyl;
R⁹ and R¹⁰ each independently represent hydrogen, C₁-C₆ alkyl or C₃-C₆ cycloalkyl;
R¹¹ and R¹² each independently represent hydrogen, C₁-C₆ alkyl or C₃-C₆ cycloalkyl, or R¹¹ and R¹² together with the nitrogen atom to which they are attached form a 4- to 7-membered saturated heterocyclic ring which may be optionally substituted with at least one substituent selected from hydroxyl;
R¹³ and R¹⁴ each independently represent C₁-C₆ alkyl, C₃-C₆ cycloalkyl or C₁-C₄ haloalkyl;
R¹⁵ and R¹⁶ each independently represent hydrogen, C₁-C₆ alkyl or C₃-C₆ cycloalkyl, or R¹⁵ and R¹⁶ together with the nitrogen atom to which they are attached form a 4- to 7-membered saturated heterocyclic ring which may be optionally substituted with at least one substituent selected from hydroxyl; and
R¹⁷ and R¹⁸ each independently represent hydrogen, C₁-C₆ alkyl or C₃-C₆ cycloalkyl, or R¹⁷ and R¹⁸ together with the nitrogen atom to which they are attached form a 4- to 7-membered saturated heterocyclic ring which may be optionally substituted with at least one substituent selected from hydroxyl;
   or a pharmaceutically acceptable salt or solvate thereof.

In the context of the present specification, unless otherwise stated, an alkyl substituent group or alkyl moiety in a substituent group may be linear or branched. A haloalkyl substituent group will comprise at least one halogen atom, e.g. one, two, three, four or five halogen atoms. In the ring substituted by R², R² may be attached to any suitable ring carbon atom including the carbon atom of (CH₂)_{q}. An unsaturated ring or ring system will be partially or fully unsaturated. Further, when R¹¹ and R¹² or R¹⁵ and R¹⁶ or R¹⁷ and R¹⁸ represent a 4- to 7-membered saturated heterocyclic ring, it should be understood that the only heteroatom present is the nitrogen atom to which R¹¹ and R¹² or R¹⁵ and R¹⁶ or R¹⁷ and R¹⁸ are attached.

In an embodiment of the invention, m is 0 or 1, particularly 1.

Each R¹ independently represents halogen (e.g. chlorine, fluorine, bromine or iodine), cyano, hydroxyl, C₁-C₆, preferably C₁-C₄, alkyl (e.g. methyl, ethyl, n-prbpyl, isopropyl, n-butyl, isobutyl, tert-butyl, n-pentyl or n-hexyl), C₁-C₆, preferably C₁-C₄, haloalkyl (e.g. trifluoromethyl or pentafluoroethyl), C₁-C₆, preferably C₁-C₄, alkoxy (e.g. methoxy, ethoxy, n-propoxy, isopropoxy, n-butoxy, isobutoxy, tert-butoxy, n-pentoxy or n-hexoxy), C₁-C₆, preferably C₁-C₄, alkylsulphonyl (e.g. methylsulphonyl, ethylsulphonyl, n-propylsulphonyl, isopropylsulphonyl, n-butylsulphonyl, isobutylsulphonyl, tert-butylsulphonyl, n-pentylsulphonyl or n-hexylsulphonyl) or sulphonamido.

In an embodiment of the invention, each R¹ independently represents halogen, C₁-C₆, preferably C₁-C₄, alkyl or C₁-C₆, preferably C₁-C₄, haloalkyl.

In another embodiment, each R¹ independently represents fluorine, chlorine, methyl or trifluoromethyl, particularly chlorine.

In an embodiment of the invention, X represents a bond and Y represents —CH₂—.

Each R² independently represents halogen (e.g. chlorine, fluorine, bromine or iodine), C₁-C₆, preferably C₁-C₄, alkyl (e.g. methyl, ethyl, n-propyl, isopropyl, n-butyl, isobutyl, tert-butyl, n-pentyl or n-hexyl) or C₁-C₆, preferably C₁-C₄, haloalkyl (e.g. trifluoromethyl or pentafluoroethyl).

In an embodiment of the invention, n is 0 or n is 1 and R² represents halogen, particularly fluorine.

R³ represents a saturated or, preferably, unsaturated 5- or 6- to 7-, 8-, 9- or 10-membered ring system other than phenyl, which ring system may comprise at least one ring heteroatom (e.g. one, two, three or four ring heteroatoms independently) selected from nitrogen, oxygen and sulphur, the ring system being optionally substituted with at least one substituent (e.g. one, two, three or four substituents independently) selected from halogen (e.g. chlorine, fluorine, bromine or iodine), cyano, oxo (═O), nitro, hydroxyl, carboxyl, —C(O)H, —NR⁹R¹⁰, —C(O)NR¹¹R¹², —NHC(O)R¹³, —NHSO₂R¹⁴, —SO₂NR¹⁵R¹⁶, —NHC(O)NR¹⁷R¹⁸, C₁-C₆, preferably C₁-C₄, alkyl (e.g. methyl, ethyl, n-propyl, isopropyl, n-butyl, isobutyl, tert-butyl, n-pentyl or n-hexyl),
C₁-C₆, preferably C₁-C₄, alkoxy (e.g. methoxy, ethoxy, n-propoxy, isopropoxy, n-butoxy, isobutoxy, tert-butoxy, n-pentoxy or n-hexoxy),
C₁-C₆, preferably C₁-C₄, alkylthio (e.g. methylthio, ethylthio, n-propylthio, isopropylthio, n-butylthio, isobutylthio, tert-butylthio, n-pentylthio or n-hexylthio),
C₁-C₆, preferably C₁-C₄, alkylsulphonyl (e.g. methylsulphonyl, ethylsulphonyl, n-propylsulphonyl, isopropylsulphonyl, n-butylsulphonyl, isobutylsulphbnyl, tert-butylsulphonyl, n-pentylsulphonyl or n-hexylsulphonyl),
C₁-C₆, preferably C₁-C₄, haloalkyl (e.g. trifluoromethyl or pentafluoroethyl),
C₁-C₆ alkoxyC₁-C₆ alkyl (e.g. C₁-C₄ alkoxyC₁-C₆ alkyl or C₁-C₂ alkoxyC₁-C₆ alkyl or
C₁-C₄ alkoxyC₁-C₄ alkyl or C₁-C₂ alkoxyC₁-C₂ alkyl such as methoxymethyl),
C₁-C₆, preferably C₁-C₄, alkylcarbonyl (e.g. methylcarbonyl, ethylcarbonyl, n-propylcarbonyl, isopropylcarbonyl, n-butylcarbonyl, isobutylcarbonyl, tert-butylcarbonyl, n-pentylcarbonyl or n-hexylcarbonyl), phenylcarbonyl,
C₃-C₆ cycloalkyl (cyclopropyl, cyclobutyl, cyclopentyl or cyclohexyl),
C₃-C₆ cycloalkylmethyl (cyclopropylmethyl, cyclobutylmethyl, cyclopentylmethyl or cyclohexylmethyl), and
   a saturated or unsaturated 5- to 6-membered heterocyclic ring comprising at least one ring heteroatom (e.g. one, two, three or four ring heteroatoms independently) selected from nitrogen, oxygen and sulphur (e.g. one or more of pyrrolidinyl, piperidinyl, piperazinyl, dithiolanyl, morpholinyl, tetrahydropyranyl, thiomorpholinyl, pyrazolyl, pyrazinyl, pyridazinyl, thiazolidinyl, thienyl, isoxazolyl, pyrimidinyl, thiadiazolyl, pyrrolyl, furanyl, thiazolyl, imidazolyl, triazolyl, tetrazolyl and pyridinyl, preferably thienyl, dithiolanyl and pyridinyl).

The saturated or unsaturated 5- to 10-membered ring system in R³ may be carbocylic or heterocyclic. Examples of suitable ring systems, which may be monocyclic or polycyclic (e.g. bicyclic) where the two or more rings are fused, include one or more (in any combination) of cyclopentyl, cyclohexyl, bicyclo[2.2. 1]heptyl, cyclopentenyl, cyclohexenyl, pyrrolidinyl, piperidinyl, piperazinyl, morpholinyl, thiomorpholinyl, diazabicyclo[2.2.1]hept-2-yl, naphthyl, benzofuranyl, benzothienyl, benzodioxolyl, isoquinolinyl, quinolinyl, 1,2-dihydroquinolinyl, 1,2,3,4-tetrahydroquinolinyl, 2,3-dihydrobenzoxazinyl, quinazolinyl, 1,2,3,4-tetrahydroquinazolinyl, 2,3-dihydrobenzofuranyl, pyrazolyl, pyrazinyl, thiazolidinyl, indanyl, thienyl, isoxazolyl, pyridazinyl, thiadiazolyl, pyrrolyl, furanyl, thiazolyl,benzothiazolyl, indolyl, imidazolyl, pyrimidinyl, benzimidazolyl, triazolyl, tetrazolyl and pyridinyl.

Preferred ring systems include quinolinyl, 1,2-dihydroquinolinyl, 1,2,3,4-tetrahydroquinolinyl, 2,3-dihydrobenzoxazinyl, 1,2,3,4-tetrahydroquinazolinyl, naphthyl, pyridinyl, benzofuranyl, benzothiazolyl, pyrimidinyl, isoquinolinyl and quinazolinyl.

In an embodiment of the invention, R³ represents an unsaturated 6- to 10-membered ring system, which ring system may comprise one, two or three ring heteroatoms independently selected from nitrogen, oxygen and sulphur, the ring system being optionally substituted with at least one substituent (e.g. one, two, three or four substituents independently) selected from halogen, cyano, oxo, nitro, hydroxyl, carboxyl, —C(O)H, —NR⁹R¹⁰, —C(O)NR¹¹R¹², —NHC(O)R¹³, —NHSO₂R¹⁴, —SO₂NR¹⁵R¹⁶, —NHC(O)NR¹⁷R¹⁸, C₁-C₄ alkyl, C₁-C₄ alkoxy, C₁-C₄ alkylthio, C₁-C₄ alkylsulphonyl, C₁-C₄ haloalkyl, C₁-C₄ alkoxyC₁-C₄ alkyl, C₁-C₄ alkylcarbonyl, phenylcarbonyl, C₃-C₆ cycloalkyl, C₃-C₆ cycloalkylmethyl and a saturated or unsaturated 5- to 6-membered heterocyclic ring comprising one or two ring heteroatoms independently selected from nitrogen, oxygen and sulphur.

In another embodiment of the invention, R³ represents an unsaturated 6- to 10-membered ring system, which ring system may comprise one or two ring heteroatoms independently selected from nitrogen and oxygen (e.g. quinolinyl, 1,2-dihydroquinolinyl, 1,2,3,4-tetrahydroquinolinyl, 2,3-dihydrobenzoxazinyl, 1,2,3,4-tetrahydroquinazolinyl, naphthyl, pyridinyl, benzofuranyl, pyrimidinyl, isoquinolinyl and quinazolinyl), or two ring heteroatoms consisting of nitrogen and sulphur (e.g. benzothiazolyl), the ring system being optionally substituted with one, two or three substituents independently selected from halogen, oxo, nitro, —NH₂, C₁-C₄ alkyl, C₁-C₄ alkoxy, C₁-C₄ alkylthio, C₁-C₄ haloalkyl, C₁-C₄ alkoxyC₁-C₄ alkyl, C₁-C₄ alkylcarbonyl, C₃-C₆ cycloalkylmethyl, —C(O)NR¹¹R¹², carboxyl and a saturated or unsaturated 5- to 6-membered heterocyclic ring comprising one or two ring heteroatoms independently selected from nitrogen and sulphur (e.g. thienyl, dithiolanyl and pyridinyl).

In a further embodiment of the invention, R³ represents an unsaturated 6- to 10-membered ring system selected from quinolinyl, 1,2-dihydroquinolinyl, 1,2,3,4-tetrahydroquinolinyl, 2,3-dihydrobenzoxazinyl, 1,2,3,4-tetrahydroquinazolinyl, naphthyl, pyridinyl, benzofuranyl, benzothiazolyl, pyrimidinyl, isoquinolinyl and quinazolinyl, the ring system being optionally substituted with one, two or three substituents independently selected from chlorine, bromine, iodine, oxo, nitro, —NH₂, C₁-C₄ alkyl, methoxy, methylthio, trifluoromethyl, methoxymethyl, methylcarbonyl, cyclopropylmethyl, carboxyl, thienyl, dithiolanyl, pyridinyl, and C(O)NR¹¹R¹² where R¹¹ represents hydrogen and R¹² represents methyl or R¹¹ and R¹² together with the nitrogen form a pyrrolidinyl group substituted by hydroxyl.

R⁴, R⁵, R⁶, R⁷ and R⁸ each independently represent hydrogen, halogen (e.g. chlorine, fluorine, brornine or iodine), C₁-C₆, preferably C₁-C₄, alkyl (e.g. methyl, ethyl, n-propyl, isopropyl, n-butyl, isobutyl, tert-butyl, n-pentyl or n-hexyl) or C₁-C₆, preferably C₁-C₄, haloalkyl (e.g. trifluoromethyl or pentafluoroethyl).

In an embodiment of the invention, R⁴, R⁵, R⁶, R⁷ and R⁸ each independently represent a hydrogen atom or a methyl group.

In another embodiment of the invention, R⁴, R⁵, R⁶ and R⁷ each represent a hydrogen atom and R⁸ represents a methyl group.

In an embodiment of the invention, R⁴, R⁵, R⁶, R⁷ and R⁸ each represent a hydrogen atom.

R⁹ and R¹⁰ each independently represent hydrogen, C₁-C₆, preferably C₁-C₄, alkyl (e.g. methyl, ethyl, n-propyl, isopropyl, n-butyl, isobutyl, tert-butyl, n-pentyl or n-hexyl) or C₃-C₆, preferably C₃ or C₅-C₆, cycloalkyl (cyclopropyl, cyclobutyl, cyclopentyl or cyclohexyl).

In an embodiment of the invention, R⁹ and R¹⁰ each represent hydrogen.

R¹¹ and R¹² each independently represent hydrogen, C₁-C₆, preferably C₁-C₄, alkyl (e.g. methyl, ethyl, n-propyl, isopropyl, n-butyl, isobutyl, tert-butyl, n-pentyl or n-hexyl) or C₃-C₆, preferably C₃ or C₅-C₆, cycloalkyl (cyclopropyl, cyclobutyl, cyclopentyl or cyclohexyl), or R¹¹ and R¹² together with the nitrogen atom to which they are attached form a 4- to 7-membered saturated heterocyclic ring (e.g. pyrrolidinyl or piperidinyl) which may be optionally substituted with at least one substituent (e.g. one, two or three substituents independently) selected from hydroxyl.

In an embodiment of the invention, R¹¹ and R¹² each independently represent hydrogen, C₁-C₄ alkyl or C₃ or C₅-C₆ cycloalkyl, or R¹¹ and R¹² together with the nitrogen atom to which they are attached form a 5- to 6-membered saturated heterocyclic ring which may be optionally substituted with one or two hydroxyl groups.

In another embodiment, R¹¹ and R¹² each independently represent hydrogen, C₁-C₂ alkyl or C₃ or C₅-C₆ cycloalkyl, or R¹¹ and R¹² together with the nitrogen atom to which they are attached form a 5-membered saturated heterocyclic ring which may be optionally substituted with one hydroxyl group.

R¹³ and R¹⁴ each independently represent C₁-C₆, preferably C₁-C₄, alkyl (e.g. methyl, ethyl, n-propyl, isopropyl, n-butyl, isobutyl, tert-butyl, n-pentyl or n-hexyl, particularly methyl), C₃-C₆, preferably C₃ or C₅-C₆, cycloalkyl (cyclopropyl, cyclobutyl, cyclopentyl or cyclohexyl) or C₁-C₄, preferably C₁-C₂, haloalkyl (e.g. trifluoromethyl or pentafluoroethyl).

R¹⁵ and R¹⁶ each independently represent hydrogen, C₁-C₆, preferably C₁-C₄, alkyl (e.g. methyl, ethyl, n-propyl, isopropyl, n-butyl, isobutyl, tert-butyl, n-pentyl or n-hexyl) or C₃-C₆, preferably C₃ or C₅-C₆, cycloalkyl (cyclopropyl, cyclobutyl, cyclopentyl or cyclohexyl), or R¹⁵ and R¹⁶ together with the nitrogen atom to which they are attached form a 4- to 7-membered saturated heterocyclic ring (e.g. pyrrolidinyl or piperidinyl) which may be optionally substituted with at least one substituent (e.g., one, two or three substituents independently) selected from hydroxyl.

R¹⁷ and R¹⁸ each independently represent hydrogen, C₁-C₆, preferably C₁-C₄, alkyl (e.g. methyl, ethyl, n-propyl, isopropyl, n-butyl, isobutyl, tert-butyl, n-pentyl or n-hexyl) or C₃-C₆, preferably C₃ or C₅-C₆, cycloalkyl (cyclopropyl, cyclobutyl, cyclopentyl or cyclohexyl), or R¹⁷ and R¹⁸ together with the nitrogen atom to which they are attached form a 4- to 7-membered saturated heterocyclic ring (e.g. pyrrolidinyl or piperidinyl) which may be optionally substituted with at least one substituent (e.g. one, two or three substituents independently) selected from hydroxyl.

In an embodiment of the invention:
m is 1;
R¹ represents halogen particularly chlorine);
X represents a bond;
Y represents —CH₂—;
n is 0;
q is 1;
R³ represents an unsaturated 6- to 10-membered ring system selected from quinolinyl, 1,2-dihydroquinolinyl, 1,2,3,4-tetrahydroquinolinyl, 2,3-dihydrobenzoxazinyl, 1,2,3,4-tetrahydroquinazolinyl, naphthyl, pyridinyl, benzofuranyl, pyrimidinyl, isoquinolinyl, benzothiazolyl and quinazolinyl, the ring system being optionally substituted with one, two or three substituents independently selected from chlorine, bromine, iodine, oxo, nitro, C₁-C₄ alkyl, methoxy, methylthio, trifluoromethyl, methoxymethyl, methylcarbonyl, cyclopropylmethyl, thienyl, dithiolanyl, —NH₂, carboxyl, pyridinyl, and C(O)NR¹¹R¹² where R¹¹ represents hydrogen and R¹² represents methyl or R¹¹ and R¹² together with the nitrogen form a pyrrolidinyl group substituted by hydroxyl; and
R⁴, R⁵, R⁶, R⁷ and R⁸ each independently represent hydrogen.

Examples of compounds of the invention include:
8-{[(2S)-3-(5-Chloro-1′H,3H-spiro[1-benzofuran-2,4′-piperidin]-1′-yl)-2-hydroxypropyl]oxy}-3,4-dihydroquinolin-2(1H)-one,
8-{[(2S)-3-(5-Chloro-1′H,3H-spiro[1-benzofuran-2,4′-piperidin]-1′-yl)-2-hydroxypropyl]oxy}quinolin-2(1H)-one,
5-{[(2S)-3-(5-Chloro-1′H,3H-spiro[1-benzofuran-2,4′-piperidin]-1′-yl)-2-hydroxypropyl]oxy}-2H-1,4-benzoxazin-3(4H)-one,
8-{[(2S)-3-(5-Chloro-1′H,3H-spiro[1-benzofuran-2,4′-piperidin]-1′-yl)-2-hydroxypropyl]oxy}quinazoline-2,4(1H,3H)-dione trifluoroacetate (salt),
(2S)-1-(5-Chloro-1′H,3H-spiro[1-benzofuran-2,4′-piperidin]-1′-yl)-3-(1-naphthyloxy)propan-2-ol trifluoroacetate (salt),
(2S)-1-(5-Chloro-1′H,3H-spiro[1-benzofuran-2,4′-piperidin]-1′-yl)-3-[(6-methyl-2-nitropyridin-3-yl)oxy]propan-2-ol trifluoroacetate (salt),
1-(6-{[(2S)-3-(5-Chloro-1′H,3H-spiro[1-benzofuran-2,4′-piperidin]-1′-yl)-2-hydroxypropyl]oxy}-4,7-dimethoxy-1-benzofuran-5-yl)ethanone trifluoroacetate (salt),
(2S)-1-[(6-Chloropyridin-2-yl)oxy]-3-(5-chloro-1′H,3H-spiro[1-benzofuran-2,4′-piperidin]-1′-yl)propan-2-ol trifluoroacetate (salt),
(2S)-1-(5-Chloro-1′H,3H-spiro[1-benzofuran-2,4′-piperidin]-1′-yl)-3-{[7-(trifluoromethyl)quinolinyl]oxy}propan-2-ol trifluoroacetate (salt),
(2S)-1-(5-Chloro-1′H,3H-spiro[1-benzofuran-2,4′-piperidin]-1′-yl)-3-[(2-iodo-6-methylpyridin-3-yl)oxy]propan-2-ol trifluoroacetate (salt),
(2S)-1-(5-Chloro-1′H,3H-spiro[1-benzofuran-2,4′-piperidin]-1′-yl)-3-{[5-(cyclopropylmethyl)-6-methyl-2-pyridin-4-ylpyrimidin-4-yl]oxy}propan-2-ol trifluoroacetate (salt),
(2S)-1-(5-Chloro-1′H,3H-spiro[1-benzofuran-2,4′-piperidin]-1′-yl)-3-(quinolin-8-yloxy)propan-2-ol trifluoroacetate (salt),
(2S)-1-(5-Chloro-1′H,3H-spiro[1-benzofuran-2,4′-piperidin]-1′-yl)-3-(isoquinolin-5-yloxy)propan-2-ol trifluotoacetate (salt),
(2)-1-[(6-Bromoquinazolin-4-yl)oxy]-3-5-chloro-1′H,3H-spiro[1-benzofuran-2,4′-piperidin]-1′-yl)propan-2-ol trifluoroacetate (salt),
(2S)-1-(5-Chloro-1′H,3H-spiro[1-benzofuran-2,4′-piperidin]-1′-yl)-3-{[2-(2-thienyl)-6-(trifluoromethyl)pyrimidin-4-yl]oxy}propan-2-ol trifluoroacetate (salt),
(2S)-1-(5-Chloro-1′H,3H-spiro[1-benzofuran-2,4′-piperidin]-1′-yl)-3-(quinolin-5-yloxy)propan-2-ol trifluoroacetate (salt),
(2S)-1-(5-Chloro-1′H,3H-spiro[1-benzofuran-2,4′-piperidin]-1′-yl)-3-[(2,3,4-trichloro-1-naphthyl)oxy]propan-2-ol trifluoroacetate (salt),
(2S)-1-(5-Chloro-1′H,3H-spiro[1-benzofuran-2,4′-piperidin]-1′-yl)-3-{[1-(1,3-dithiolan-2-yl)-2-naphthyl]oxy}propan-2-ol trifluoroacetate (salt),
(2S)-1-{[5-Butyl-6-(methoxymethyl)-2-(methylthio)pyrimidin-4-yl]oxy}-3-(5-chloro-1′H,3H-spiro[1-benzofuran-2,4′-piperidin]-1′-yl)propan-2-ol trifluoroacetate (salt),
(2S)-1-[(2-Amino-1,3-benzothiazol4-yl)oxy]-3-(5-chloro-1′H,3H-spiro[1-benzofuran-2,4′-piperidin]-1′-yl)propan-2-ol,
(2S)-1-(5-Chloro-1′H,3H-spiro[1-benzofuran-2,4′-piperidin]-1′-yl)-3-[(2-methyl-1,3-benzothiazol-4-yl)oxy]propan-2-ol,
(2S)-1-(5-chloro-1′H,3H-spiro[1-benzofuran-2,4′-piperidin]-1′-yl)-3-[(2-methyl-1-benzofuran-4-yl)oxy]propan-2-ol,
3-{[(2S)-3-(5-chloro-1′H,3H-spiro[1-benzofuran-2,4′-piperidin]-1′-yl)-2-hydroxypropyl]oxy}isonicotinic acid,
3-{[(2S)-3-(5-Chloro-1′H,3H-spiro[1-benzofuran-2,4′-piperidin]-1′-yl)-2-hydroxypropyl]oxy}-N-methylisonicotinamide,
(3S)-1-(3-{[(2S)-3-(5-Chloro-1′H,3H-spiro[1-benzofuran-2,4′-piperidin]-1′-yl)-2-hydroxypropyl]oxy}isonicotinoyl)pyrrolidin-3-ol,
   and pharmaceutically acceptable salts and solvates of any one thereof.

The present invention further provides a process for the preparation of a compound of formula (I) or a pharmaceutically acceptable salt or solvate thereof as defined above which comprises,
(a) reacting a compound of formula 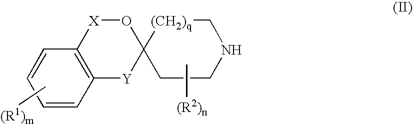
   wherein m, R¹, n, R², q, X and Y are as defined in formula (I), with a compound of formula 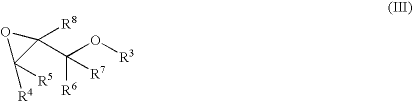
   wherein R³, R⁴, R⁵, R⁶, R⁷ and R⁸ are as defined in formula (I); or
(b) reacting a compound of formula 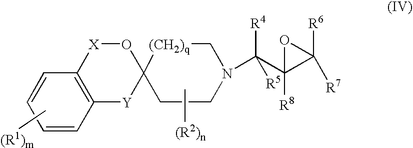
   wherein m, R¹, n, R², q, X, Y, R⁴, R⁵, R⁶, R⁷ and R⁸ are as defined in formula (I), with a compound of formula
   HO—R³ (V)
   wherein R³ is as defined in formula (I), in the presence of a suitable base (for example, triethylamine or potassium carbonate);
(c) when R³ is substituted with —C(O)NR¹¹R¹², reacting a compound of formula 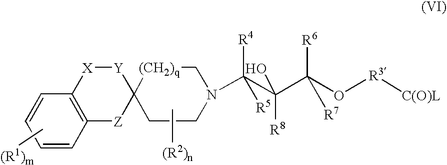
   wherein L represents a leaving group (e.g. a hydroxyl group), R^{3′} is a saturated or unsaturated 5- to 10-membered ring system other than phenyl, which ring system may comprise at least one ring heteroatom selected from nitrogen, oxygen and sulphur, [and which ring system may be further substituted with a substituent other than —C(O)NR¹¹R¹² as defined for R3 in formula (I)] and m, R¹, n, R², q, X, Y, Z, R⁴, R⁵, R⁶, and R⁷ are as defined in formula (I), with a compound of formula (VII),
   NHR¹¹R¹² (VII)
   wherein R¹¹ and R¹² are as defined in formula (I), in the presence of a suitable coupling reagent (e.g. ethyl chloridocarbonate or 1,1′-carbonylbis-1H-imidazole);
   and optionally after (a), (b) or (c) forming a pharmaceutically acceptable salt or solvate.

The processes of the invention may conveniently be carried out in a solvent, e.g. an organic solvent such as an alcohol (e.g. methanol or ethanol), a hydrocarbon (e.g. toluene) or tetrahydrofuran, dimethylformamide, N-methylpyrrolidinone, dichloromethane or acetonitrile at a temperature of, for example, 0° C. or above such as a temperature in the range from 0, 5, 10, 15 or 20° C. to 100, 110 or 120° C.

Compounds of formulae (II), (III), (IV), (V) and (VII) are either commercially available, are known in the literature or may be prepared using known techniques.

Compound (VI) can be prepared according to the general processes described in process (a) and process (b).

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in is Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) above may be converted to a pharmaceutically acceptable salt or solvate thereof, preferably an acid addition salt such as a hydrochloride, hydrobromide, phosphate, acetate, fumarate, maleate, tartrate, citrate, oxalate, methanesulphonate or p-toluenesulphonate.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired.

The compounds of formula (I) have activity as pharmaceuticals, in particular as modulators of chemokine receptor (especially MIP-1α chemokine receptor) activity, and may be used in the treatment of autoimmune, inflammatory, proliferative and hyperproliferative diseases and immunologically-mediated diseases including rejection of transplanted organs or tissues and Acquired Immunodeficiency Syndrome (AIDS).

Examples of these conditions are:
(1) (the respiratory tract) airways diseases including chronic obstructive pulmonary disease (COPD) such as irreversible COPD; asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia;
(2) (bone and joints) rheumatoid arthritis, seronegative spondyloarthropathies (including ankylosing spondylitis, psoriatic arthritis and Reiter's disease), Behcet's disease, Sjogren's syndrome and systemic sclerosis;
(3) (skin) psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis;
(4) (gastrointestinal tract) Coeliac disease, proctitis, eosinopilic gastro-enteritis, mastocytosis, Crohn's disease, ulcerative colitis, food-related allergies which have effects remote from the gut, e.g., migraine, rhinitis and eczema;
(5) (other tissues and systemic disease) multiple sclerosis, atherosclerosis, Acquired Immunodeficiency Syndrome (AIDS), lupus erythematosus, systemic lupus, erythematosus, Hashimoto's thyroiditis, myasthenia gravis, type I diabetes, nephrotic syndrome, eosinophilia fascitis, hyper IgE syndrome, lepromatous leprosy, sezary syndrome and idiopathic thrombocytopenia pupura;
(6) (allograft rejection) acute and chronic following, for example, transplantation of kidney, heart, liver, lung, bone marrow, skin and cornea; and chronic graft versus host disease;
(7) cancers, especially non-small cell lung cancer (NSCLC) and squamous sarcoma;
(8) diseases in which angiogenesis is associated with raised chemokine levels; and
(9) cystic fibrosis, stroke, re-perfusion injury in the heart, brain, peripheral limbs and sepsis.

Thus, the present invention provides a compound of formula (I), or a pharmaceutically-acceptable salt or solvate thereof, as hereinbefore defined for use in therapy.

In a further aspect, the present invention provides the use of a compound of formula (I), or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

The invention also provides a method of treating an inflammatory disease (e.g. rheumatoid arthritis) which comprises administering to a patient in need thereof a therapeutically effective amount of a compound of formula (I), or a pharmaceutically acceptable salt or solvate thereof, as hereinbefore defined.

The invention still further provides a method of treating an airways disease (e.g. asthma or chronic obstructive pulmonary disease) which comprises administering to a patient in need thereof a therapeutically effective amount of a compound of formula (I), or a pharmaceutically acceptable salt or solvate thereof, as hereinbefore defined.

For the above-mentioned therapeutic uses the dosage administered will, of course, vary with the compound employed, the mode of administration, the treatment desired and the disorder indicated. The daily dosage of the compound of formula (I) may be in the range from 0.001 mg/kg to 30 mg/kg.

The compounds of formula (I) and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt or solvate thereof, as hereinbefore defined, in association with a pharmaceutically acceptable adjuvant, diluent or carrier.

The invention further provides a process for the preparation of a pharmaceutical composition of the invention which comprises mixing a compound of formula (I), or a pharmaceutically acceptable salt or solvate thereof, as hereinbefore defined, with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane aerosols and dry powder formulations; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

The invention will now be further explained by reference to the following illustrative examples, in which ¹H NMR spectra were recorded on Varian Unity Inova 400. The central solvent peak of chloroform-d (δ_{H} 7.27 ppm), acetone-d₆ (δ_{H} 2.05 ppm), DMSO-d₆ (δ_{H} 2.50 ppm), or methanol-d₄ (δ_{H} 4.87 ppm) were used as internal standard. Low resolution mass spectra and accurate mass determination were recorded on a Hewlett-Packard 1100 LC-MS system equipped with APCI/ESI ionisation chambers. All solvents and commercial reagents were laboratory grade and used as received. The nomenclature used for the compounds was generated with ACD/Name and ACD/Name Batch. The abbreviations or terms used in the examples have the following meanings:
DMF: N,N-dimethylformamide
MeOH: methanol
DCM: dichloromethane
THF: tetrahydrofiran
DME: 1,2-dimethoxyethane
NMP: N-methylpyrrolidinone

### EXAMPLES

### Intermediate Compound: 5-Chloro-3H-spiro[1-benzofuran-2,4′-piperidine]

Method A: This compound was prepared as described by Effland, R. C; Gardner, B. A; Strupczewski, J., J. Heterocyclic Chem., 1981, 18, 811-814.

Method B:

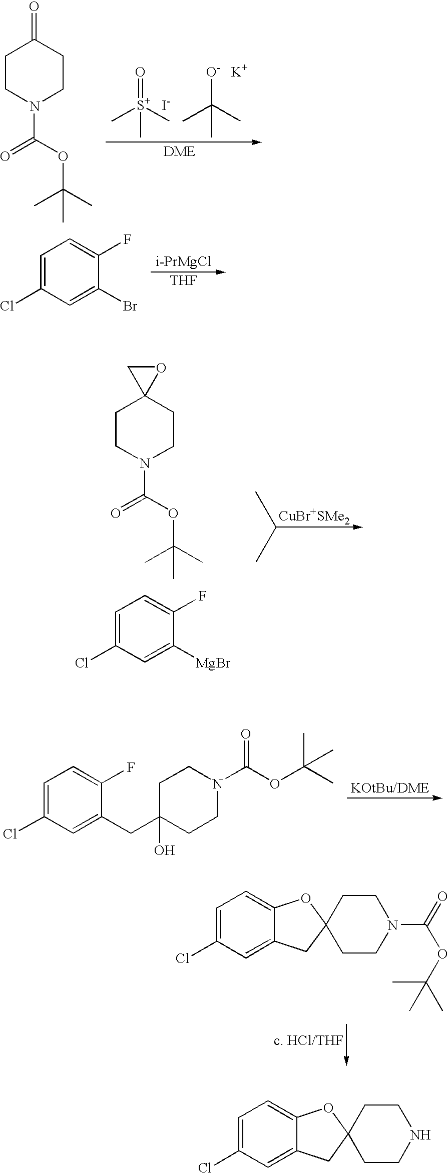

### i) 1-Oxa-6-azaspiro[2.5]octane-6-carboxylic acid, 1,1-dimethylethyl ester

Potassium t-butoxide (31 g) was added to a stirred suspension of trimethylsulfoxonium iodide (60.8 g) in 1,2-dimethoxyethane (250 ml) at 20° C. After 1 hour, the mixture was added portionwise over 30 minutes to a stirred solution of 4-oxo-1-piperidinecarboxylic acid, 1,1-dimethylethyl ester (50 g) in 1,2-dimethoxyethane (50 ml) at 0° C. After a further 2 hours, water (500 ml) was added and the mixture extracted with tert.-butyl methyl ether (2×500 ml). The organic extracts were washed separately with saturated sodium bicarbonate solution (250 ml), combined, dried over anhydrous magnesium sulphate, filtered and evaporated under reduced pressure. The residual oil was co-evaoprated with toluene (100 ml) to give the sub-title compound (43.25 g, 81%) as a solid.

¹H-NMR (400 MHz, CDCl₃): δ 1.46 (9H, s), 1.43-1.48 (2H, m), 1.75-1.84 (2H, m), 2.69 (2H, s), 3.38-3.47 (2H, m), 3.70-3.75 (2H, m).

### (ii) 5-Chlorospiro[1-benzofuran-2,4′-piperidine]-1′-carboxylic acid, 1,1-dimethyl ester

A solution of iso-propylmagnesium chloride in tetrahydrofuran (2M, 106.6 ml) was added dropwise over 15 minutes to a stirred solution of 2-bromo-4-chloro-1-fluorobenzene (42.5 g) in anhydrous tetrahydrofuran (250 ml) at 0° C. under nitrogen. After a further 15 minutes, a solution of 1-oxa-6-azaspiro[2.5]octane-6-carboxylic acid, 1,1-dimethylethyl ester (43.2 g) in anhydrous tetrahydrofuran (50 ml) was added followed by copper(I)bromide dimethyl sulphide complex (0.4 g). The mixture was stirred at 40° C. for 18 hours, cooled to 20° C., diluted with water (300 ml) and extracted with tert.-butyl methyl ether (2×300 ml). Organic extracts were dried over anhydrous magnesium sulphate, filtered and evaporated under reduced pressure. The residual oil was dissolved in 1,2-dimethoxypropane (200 ml). Potassium tert-butoxide (22.8 g) was added and the mixture stirred at 40° C. for 16 hours then at 50° C. for 24 hours. Further potassium tert.-butoxide (5.7 g) was added and stirring continued at 50° C. for 2 hours then at 55° C. for 4 hours. Water (500 ml) was added and the mixture extracted with tert.-butyl methyl ether (2×300 ml). Organic extracts were dried over anhydrous magnesium sulphate, filtered and evaporated under reduced pressure to give the sub-title compound (47.45 g, 67%) as an oil.

¹H-NMR (400 MHz, CDCl₃): δ 1.47 (9H, s), 1.67 (2H, td), 1.85-1.93 (2H, m), 2.94 (2H, s), 3.39 (2H, td), 3.65-3.80 (2H, m), 6.67 (1H, d), 7.06 (1H, d), 7.10 (1H, s).

### iii) 5-Chlorospiro[1-benzofuran-2,4′-piperidine]

Concentrated hydrochloric acid (23 ml) was added to a solution of 5-chlorospiro[1-benzofuran-2,4′-piperidine]-1′-carboxylic acid, 1,1-dimethyl ester (46.43 g) in tetrahydrofuran (230 ml). The mixture was stirred at 50° C. for 6 hours, cooled to 20° C., diluted with water (230 ml) and extracted with tert.-butyl methyl ether (2×230 ml). The aqueous phase was adjusted to pH>10 by addition of 50 wt. % sodium hydroxide solution and extracted with tert.-butyl methyl ether (3×300 ml). Organic extracts were dried over anhydrous magnesium sulphate, filtered and evaporated under reduced pressure. The residual oil was dissolved in tetrahydrofuran (240 ml), concentrated hydrochloric acid (12 ml) was added and the mixture stirred at 20° C. for 16 hours. Precipitated solid was filtered and dissolved in water (100 ml). The solution was adjusted to pH>10 by addition of 50 wt. % sodium hydroxide solution and extracted with tert.-butyl methyl ether (3×100 ml) to give the title compound (13.3 g, 45%) as a solid.

¹H-NMR (400 MHz, CDCl₃): δ 1.69-1.76 (2H, m), 1.83-1.87 (2H, m), 2.78-2.84 (2H, m), 2.98-3.03 (4H, m), 6.65 (1H, d), 7.04 (1H, d), 7.13 (1H, s).

APCI-MS: m/z 224/6 [M+H]⁺

### Example 1

### 8-{[(2S)-3-(5-Chloro-1′H,3H-spiro[1-benzofuran-2,4′-piperidin]-1′-yl)-2-hydroxypropyl]oxy}-3,4-dihydroquinolin-2(1H)-one

Step I:

### 3-Chloro-N-(2-hydroxyphenyl)propanamide

T a stirred solution of 2-aminophenol (2.18 g, 20 mmol) in acetone (20 ml) was added dropwise a solution of 3-chioropropanoyl chloride (1.28 g, 10 mmol) in acetone (20 ml). The mixture was stirred for 30 min, then water (50 ml) was added. Acetone was removed in vacuo. The precipitate was collected by filtration and dried to afford subtitle compound (1.53 g, 77%).

¹H-NMR (400 MHz, DMSO-d₆): δ 9.75 (s, 1H), 9.37 (s, 1H), 7.77 (d, J=7.8 Hz, 1H), 6.91 (m, 2H), 6.76 (t, J=7.5 Hz, 1H), 3.86 (t, J=6.2 Hz, 2H), 2.91 (t, J=6.2 Hz, 2H).

APCI-MS: m/z 200 (MH⁺).

Step II:

### 8-Hydroxy-3,4-dihydroquinolin-2(1H)-one

A mixture of 3-chloro-N-(2-hydroxyphenyl)propanamide (0.25 g, 1.25 mmol) and AlCl₃ (0.5 g) was heated with stirring at 130° C. for 5 h. After cooling to room temperature, the reaction mixture was quenched with water (3 ml). The resulting suspension was extracted with ethyl acetate (3×5 ml). Evaporation of solvent from combined extracts and purification by flash chromatography on silica gel (ethyl acetate/heptane) afforded colourless crystals (95 mg, 47%).

¹H-NMR (400 Mz, DMSO-d₆): δ 9.64 (s, 1H), 8.76 (s, 1H), 6.75 (m, 1H), 6.65 (m, 2H), 2.83 (t, J=7.4 Hz, 2H), 2.43 (t, J=7.5 Hz, 2H).

APCI-MS: m/z 164 (MH⁺).

Step III:

### 5-Chloro-1′-[(2S)-oxiran-2-ylmethyl]-3H-spiro[1-benzofuran-2,4′-piperidine]

A solution of 5-chloro-3H-spiro[1-benzofuran-2,4′-piperidine] (0.22 g, 1 mmol) and (2R)-2-(chloromethyl)oxirane (92 mg, 1 mmol) in dry ethanol (5 ml) was stirred at room temperature for 36 hours. A solution of sodium methoxide in methanol (0.5 M, 2 ml) was added dropwise, and the stirring was continued at room temperature for 1 hour. The inorganic precipitate was removed by filtration. The solvent was removed in vacuo, and the residue purified by flash chromatography on silica gel (dichloromethane/methanol, 1:1) to afford a colourless oil (0.20 g, 71%).

¹H-NMR (400 MHz, CDCl₃): δ 7.09 (s, 1H), 7.05 (ddd, J=0.2, 8.2, 2.0 Hz, 2H), 6.66 (d, J=8.5 Hz, 1H), 3.13 (sextet, J=3.4 Hz, 1H), 2.97 (s, 2H), 2.79 (m, 2H), 2.75-2.57 (m, 4H), 2.51 (dd, J=5.0, 2.7 Hz, 1H), 2.36 (dd, J=13.4, 6.7 Hz, 1H), 1.99 (m, 2H), 1.84 (m, 2H).

APCI-MS: m/z 280 (MH⁺).

Step IV:

### 8-{[(2S)-3-(5-Chloro-1′H,3H-spiro[1-benzofuran-2,4′-piperidin]-1′-yl)-2-hydroxypropyl]oxy}-3,4dihydroquinolin-2(1H)-one

A mixture of 8-hydroxy-3,4-dihydroquinolin-2(1H)-one (25 mg, 0.16 mmol), 5-chloro-1′-[(2S)-oxiran-2-ylmethyl]-3H-spiro[1-benzofuran-2,4′-piperidine] (40 mg, 0.14 mmol), and K₂CO₃ (30 mg, 0.22 mmol) in DMF (1 ml) was stirred at 110° C. for 24 hours. After cooling to room temperature, the inorganic material was removed by filtration. The filtrate was concentrated in vacuo. Purification by semi-preparative HPLC yielded the title compound (16 mg, 25%).

¹H-NMR (400 MHz, DMSO-d₆): δ 9.50 (s, 1H), 7.23 (s, 1H), 7.09 (dd, J=8.4, 2.1 Hz, 1H), 6.91-6.71 (m, 4H), 5.30 (d, J=5.2 Hz, 1H), 4.01 (d, J=7.2 Hz, 2H), 3.80 (dd, J=9.8, 7.4 Hz, 1H), 2.99 (s, 2H), 2.87 (t, J=7.5 Hz, 2H), 2.65 (m, 1H), 2.60-2.41 (m, 6H), 1.86-1.68 (m, 4H)

APCI-MS: m/z 443 (MH⁺).

### Example 2

### 8-{[(2S)-3-(5-Chloro-1′H,3H-spiro[1-benzofuran-2,4′-piperidin]-1-yl)-2-hydroxypropyl]oxy}quinolin-2(1H)-one

Step I:

### 8-Hydroxyquinolin-2(1H)-one

Quinolin-8-ol 1-oxide (20 g, 124 mmol) in acetic anhydride (200 ml) was stirred at 90° C. for 5 hours. Then the reaction mixture was poured into water/ice mixture (1.5 L), and made neutral by addition of conc. aq. NH₃. The precipitate formed was collected by filtration and washed with water. The crude product was purified by suspending in propan-2-ol and addition of petroleum ether to give 2-oxo-1,2-dihydroquinolin-8-yl acetate. 2-Oxo-1,2-dihydroquinolin-8-yl acetate was heated in conc. aq. HCl (200 ml) at 90° C. for 4 hours. The reaction mixture was poured into ice-cold water (400 ml), and the precipitate formed was collected by filtration and washed with water. Recrystallization from propan-2-ol/petroleum ether afforded the subtitle compound (14.1 g, 70%).

¹H-NMR (400 MHz, DMSO-d₆): δ 10.46 (s, 1H), 10.21 (s, 1H), 7.84 (d, J=9.5 Hz, 1H), 7.10 (d, J=7.6 Hz, 1H), 7.00 (t, J=7.7 Hz, 1H), 6.95 (dd, J=7.8, 1.2 Hz, 1H), 6.48 (d, J=9.5Hz, 1H)

APCI-MS: m/z 162 (MH⁺).

Step II:

### 8-{[(2S)-3-(5-Chloro-1′H,3H-spiro[1-benzofuran-2,4′-piperidin]-1′-yl)-2-hydroxypropyl]oxy}quinolin-2(1H)-one

The title compound was prepared from 8-hydroxyquinolin-2(1H)-one (28 mg, 0.1 mmol) using the procedure described in Example 1, Step IV. Purification by preparative HPLC afforded 44 mg (38%).

¹H-NMR (400 MHz, DMSO-d₆): δ 11.17 (s, 1H), 7.90 (d, J=9.5 Hz, 1H), 7.25 (m, 2H), 7.10 (m, 3H), 6.74 (d, J=8.5 Hz, 1H), 6.53 (d, J=9.6 Hz, 1H), 5.45 (d, J=5.5 Hz, 1H), 4.14 (dd, J=9.2, 2.5 Hz, 1H), 4.09 (t, J=5.9 Hz, 1H), 3.92 (dd, J=9.0, 6.8 Hz, 1H), 3.00 (s, 2H), 2.67 (br.s, 1H), 2.60-2.41 (m, 5H, partially civered with the signal of solvent), 1.86-1.69 (m, 4H)

APCI-MS: m/z 441 (MH⁺).

### Example 3

### 5-{[(2S)-3-(5-Chloro-1′H,3H-spiro[1-benzofuran-2,4′-piperidin]-1′-yl)-2-hydroxypropyl] oxy}-2H-1,4-benzoxazin-3(4H)-one

Step I:

### 2-Aminobenzene-1,3-diol

A mixture of 2-nitrobenzene-1,3-diol (5 g, 32.2 mmol) and 10% Pd on charcoal (230 mg) in ethanol (100 ml) were hydrogenated with H₂ at atmospheric pressure overnight. The reaction mixture was filtered through celite. Ethanol was removed by evaporation to yield the subtitled compound (4 g, 99%).

¹H-NMR (400 MHz, DMSO-d₆): δ 8.81 (br.s, 2H), 6.24 (m, 3H), 3.81 (br.s, 2H).

APCI-MS: m/z 126.0 (MH⁺).

Step II:

### 2-Chloro-N-(2,6-dihydroxyphenyl)acetamide

KH₂PO₄ (17.2 g, 126.3 mmol) and K₂HPO₄ (8.2 g, 35.7 mmol) in 188 ml of distilled water were deoxygenated by passing argon through the mixture for 0.5 hour. 2-Aminobenzene-1,3-diol (1 g, 8.0 mmol) was added to the buffer solution and chloroacetyl chloride (0.64 ml, 8.0 mmol) was added slowly to the reaction mixture. After addition was completed, the reaction mixture was stirred at room temperature for 1.5 hours. Water was removed by freeze-drying and the residue was dissolved in 20% MeOH in DCM. The insoluble salt was removed by filtration, the solvent was evaporated to give the subtitled compound which was used without purification in the next step.

APCI-MS: m/z 202.0 (MH⁺).

Step III:

### 5-Hydroxy-2H-1,4-benzoxazin-3(4H)-one

2-Chloro-N-(2,6-dihydroxyphenyl)acetamide (1.99 g, 9.88 mmol) was dissolved in 150 ml of 10% aqueous K₂CO₃ and the solution was heated to 40° C. for 45 minutes. After cooling and neutralization with 2M HCl the reaction mixture was extracted with ethyl acetate. Drying with MgSO₄ and evaporation of solvent afforded crude material (0.54 g, overall yield from Steps II and III 41%).

APCI-MS: m/z 166.0 (MH⁺).

Step IV:

### 5-{[(2S)-3-(5-Chloro-1′H,3H-spiro[1-benzofuran-2,4′-piperidin]-1′-yl)2-hydroxypropyl]oxy}-2H-1,4-benzoxazin-3(4H)-one

A mixture of 5-hydroxy-2H-1,4-benzoxazin-3(4H)-one (64.7 mg, 0.38 mmol), 5-chloro-1′-[(2S)-oxiran-2-ylmethyl]-3H-spiro[1-benzofuran-2,4′-piperidine] (105.3 mg, 0.38 mmol), K₂CO₃ (108.7 mg, 0.75 mmol) and DMF (4 ml) was heated at 110° C. overnight. After cooling the reaction mixture was partitioned between ethyl acetate and water. The organic layer was washed with water and then evaporated. The residue was purified by preparative HPLC (eluant: [acetonitrile/water]) to afford the titled compound (8 mg, 4.6%).

¹H-NMR (400 MHz, DMSO-d₆): δ 10.23 (s, 1H), 7.25 (s, 1H), 7.09 (dd, J=8.5, 2.2 Hz, 1H), 6.87 (t, J=8.3 Hz, 1H), 6.74 (d, J=8.5 Hz, 1H), 6.66 (d, J=12.8 Hz, 1H), 6.58 (d, J=8.1 Hz, 1H), 5.20 (br.s, 1H), 4.55 (s, 2H), 4.02 (m, 2H), 3.83 (m, 1H), 3.00 (s, 2H), 2.72-2.41 (br.m, 6H, partially covered with the signal of solvent), 1.79 (m, 4H).

APCI-MS: m/z 445.2 (MH⁺).

### Example 4

### 8-{[(2S)-3-(5-Chloro-1′H,3H-spiro[1-benzofuran-2,4′-piperidin]-1′-yl)-2-hydroxypropyl]oxy}quinazoline-2,4(1H,3R)-dione trifluoroacetate (salt)

Step I:

### 8-Hydroxyquinazoline-2,4(1H,3R)-dione

2-Amino-3-hydroxybenzoic acid (195 mg, 1.28 mmol), urea (243 mg, 4.0 mmol) and NMP (10 ml) were heated in microwave oven (200° C., 250 W) for 20 minutes. The reaction mixture was purified by preparative HPLC (eluant: [acetonitrile/water/trifluoroacetic acid]) to afford the subtitled compound (65 mg, 29%)

¹H-NMR (400 Mz, DMSO-d₆): δ 11.19 (s, 1H), 10.35 (s, 1H), 10.22 (s, 1H), 7.35 (dd, J=7.8, 0.8 Hz, 1H), 7.07 (dd, J=7.8, 1.3 Hz, 1H), 6.99 (t, J=7.8 Hz, 1H).

APCI-MS: m/z 178.9 (MH⁺).

Step II:

### 8-{[(2S)-3-(5-Chloro-1′H,3H-spiro[1-benzofuran-2,4′-piperidin]-1′-yl)-2-hydroxypropyl]oxy}quinazoline-2,4(1H,3H)-dione trifluoroacetate (salt)

The title compound was prepared from 8-hydroxyquinazoline-2,4(1H,3H)-dione (65.1 mg, 0.37 mmol) using the procedure described in Example 3, Step IV. Purification by preparative HPLC (eluant: [acetonitrile/water/trifluoroacetic acid]) afforded the titled compound (6 mg, 2.9%).

¹H-NMR (400 MHz, DMSO-d₆): δ 11.40 (s, 1H), 10.42 (s, 1H), 9.52 (br.s, 1H), 7.51 (d, J=7.6 Hz, 1H), 7.30 (s, 1H), 7.29 (d, J=7.0 Hz, 1H), 7.17 (d, J=8.6 Hz, 1H), 7.15 (t, J=8.0 Hz, 1H), 6.80 (d, J=8.6 Hz, 1H), 6.17 (d, J=4.1 Hz, 1H), 4.43 (br.s, 1H), 4.11 (m, 1H), 3.96 (m, 1H), 3.64-3.17 (br.m, 6H, partially covered with the signal of solvent), 3.12 (s, 2H), 2.22-2.03 (m, 4H).

APCI-MS: m/z 458.2 (MH⁺).

### Example 5

### (2S)-1-(5-Chloro-1′H,3H-spiro[1-benzofuran-2,4′-piperidin]-1′-yl)-3-(1-naphthyloxy)propan-2-ol trifluoroacetate (salt)

A slurry of 1-naphthol (100 μL, 0.5 M in dimethylformamide), (2S)-oxiran-2-ylmethyl 3-nitrobenzenesulfonate (100 μL, 0.5 M in dirnethylformamide) and cesium carbonate (13 mg, 0.04 mmol) was stirred at room temperature overnight, and then partiotioned between water and dichloromethane. The organic phase was evaporated, and the resulting crude (2S)-2-[(1-naphthyloxy)methyl]oxirane was dissolved in ethanol (400 μL) and 5-chloro-3H-spiro[1-benzofuran-2,4′-piperidine] (100 μL, 0.5 M in dimethylformamide) was added. The mixture was refluxed overnight, and the solvent was evaporated. Purification was performed on by semi-preparative HPLC, with acetonitrile/water 0.1% trifluoroacetic acid as mobile phase. Pure fractions were collected, pooled and evaporated to give the title compound.

APCI-MS m/z: 424 [MH⁺]

The following Examples 6 to 19 were prepared by methods analogous to the method described in Example 5.

### Example 6

### (2S)-1-(5-Chloro-1′H,3H-spiro[1-benzofuran-2,4′-piperidin]-1′-yl)-3-[(6-methyl-2-nitropyridin-3-yl)oxy]propan-2-ol trifluoroacetate (salt)

APCI-MS m/z: 434 [MH⁺]

### Example 7

### 1-(6-{[(2S)-3-(5-Chloro-1′H,3H-spiro[1-benzofuran-2,4′-piperidin]-1′-yl)-2-hydroxypropyl]oxy}4,7-dimethoxy-1-benzofuran-5-yl)ethanone trifluoroacetate (salt)

APCI-MS m/z: 516 [MH⁺]

### Example 8

### (2S)-1-[(6-Chloropyridin-2-yl)oxy]-3-(5-chloro-1′H,3H-spiro[1-benzofuran-2,4′-piperidin]-1′-yl)propan-2-ol trifluoroacetate (salt)

APCI-MS m/z: 409 [MH⁺]

### Example 9

### (2S)-1-(5Chloro-1′H,3H-spiro[1-benzofuran-2,4′-piperidin]-1′-yl)-3-{[7-(trifluoromethyl)quinolin-4yl]oxy}propan-2-ol trifluoroacetate (salt)

APCI-MS m/z: 493 [MH⁺]

### Example 10

### (2S)-1-(5-Chloro-1′H,3H-spiro[1-benzofuran-2,4′-piperidin]-1′-yl)-3-[(2-iodo-6-methylpyridin-3-yl)oxy]propan-2-ol trifluoroacetate (salt)

APCI-MS m/z: 515 [MH⁺]

### Example 11

### (2S)-1-(5-Chloro-1′H,3H-spiro[1-benzofuran-2,4′-piperidin]-1′-yl)-3-{[5-(cyclopropylmethyl)-6-methyl-2-pyridin-4-ylpyrimidin-4-yl]oxy}propan-2-ol trifluoroacetate (salt)

APCI-MS m/z: 521 [MH⁺]

### Example 12

### (2S)-1-(5-Chloro-1′H,3H-spiro[1-benzofuran-2,4′-piperidin]-1′-yl)-3-(quinolin-8-yloxy)propan-2-ol trifluoroacetate (salt)

APCI-MS m/z: 425 [MH⁺]

### Example 13

### (2S)-1-(5-Chloro-1′H,3H-spiro[1-benzofuran-2,4′-piperidin]-1′-yl)-3-(isoquinolin-5 -yloxy)propan-2-ol trifluoroacetate (salt)

APCI-MS m/z: 425 [MH⁺]

### Example 14

### (2S)-1-[(6-Bromoquinazolin-4-yl)oxy]-3-(5-chloro-1′H,3H-spiro[1-benzofuran-2,4′-piperidin]-1′-yl)propan-2-ol trifluoroacetate (salt)

APCI-MS m/z: 505 [MH⁺]

### Example 15

### (2S)-1-(5-Chloro-1′H,3H-spiro[1-benzofuran-2,4′-piperidin]-1′-yl)-3-{[2-(2-thienyl)-6-(trifluoromethyl)pyrimidin-4-yl]oxy}propan-2-ol trifluoroacetate (salt)

APCI-MS m/z: 526 [MH⁺]

### Example 16

### (2S)-1-(5-Chloro-1′H,3H-spiro[1-benzofuran-2,4′-piperidin]-1′-yl)-3-(quinolin-5-yloxy)propan-2-ol trifluoroacetate (salt)

APCI-MS m/z: 425 [MH⁺]

### Example 17

### (2S)-1-(5-Chloro-1′H,3H-spiro[1-benzofuran-2,4′-piperidin]-1′-yl)-3-[(2,3,4-trichloro-1-naphthyl)oxy]propan-2-ol trifluoroacetate (salt)

APCI-MS m/z: 526 [MH⁺]

### Example 18

### (2S)-1-(5-Chloro-1′H,3H-spiro[1-benzofuran-2,4′-piperidin]-1′-yl)-3-{[1-(1,3-dithiolan-2-yl)-2-naphthyl]oxy}propan-2-ol trifluoroacetate (salt)

APCI-MS m/z: 528 [MH⁺]

### Example 19

### (2S)-1-{[5-Butyl-6-(methoxymethyl)-2-(methylthio)pyrimidin-4-yl]oxy}-3-(5-chloro-1′H,3H-spiro[1-benzofuran-2,4′-piperidin]-1′-yl)propan-2-ol trifluoroacetate (salt)

APCI-MS m/z: 524 [MH⁺]

### Example 20

### (2S)-1-[(2-Amino-1,3-benzothiazol-4yl)oxy]-3-(5-chloro-1′H,3H-spiro[1-benzofuran-2,4′-piperidin]-1′-yl)propan-2-ol

Step I:

### 2-Amino-1,3-benzothiazol-4-ol

To a cold solution (ice-water bath) of 4-methoxy-1,3-benzothiazol-2-amine (360 mg, 2 mmol) in CH₂Cl₂ (10 mL) was slowly added a solution of BBr₃ in CH₂Cl₂ (1 M, 5 mL, 5 mmol). After the addition was completed, the ice-bath was removed and the reaction mixture was stirred at room temperature for 24 h, cooled to 0° C., and quenched with methanol (3 mL). After stirring for 30 min the volatiles were removed i.vac. The residue was dissolved in ethyl acetate (100 mL), washed successively with aqueous NaHCO₃ (3×10 mL) and H₂O (10 mL). The organic layer was dried over Na₂SO₄, filtered and concentrated to give crude sub title compound (270 mg).

¹H-NMR (400 MHz, DMSO-d₆): δ 9.20 (s, 1H); 7.20 (s, 2H); 7.05 (dd, J=0.8, 7.7 Hz, 1H); 6.82 (t, J=7.8 Hz, 1H); 6.64 (dd, J=0.8, 7.8 Hz, 1H).

Step II:

### (2S)-1-[(2-Amino-1,3-benzothiazol-4-yl)oxy]-3-(5-chloro-1′H,3H-spiro[1-benzofuran-2,4′-piperidin]-1′-yl)propan-2-ol

A mixture of 2-amino-1,3-benzothiazol4-ol (100 mg, 0.6 mmol), (2S)-oxiran-2-ylmethyl-3-nitrobenzenesulfonate (156 mg, 0.6 mmol) and Cs₂CO₃ (195 mg, 0.6 mmol) in DMF (3 mL) was stirred at room temperature over night. The mixture was partitioned between ethyl acetate and H₂O. The organic layer was dried over Na₂SO₄, and filtered. 5-Chloro-3H-spiro[1-benzofuran-2,4′-piperidine] (134 mg, 0.6 mmol) was added to the filtrate, and the solution was concentrated in vacuo. The residue was taken into ethanol (3 mL) and stirred at 75° C. for 3 h. The volatiles were removed in vacuo and the residue was purified by silica gel flash chromatography (0-2% methanol in CH₂Cl₂ containing 0.2% ammonia) to give title compound (60 mg).

¹H-NMR (400 MHz, DMSO-d₆): δ 7.41 (s, 2H); 7.24 (m, 2H); 7.09 (dd, J=2.3, 8.5 Hz, 1H); 6.94 (t, J=7.9 Hz, 1H); 6.84 (d, J=8.1 Hz, 1H); 6.74 (d, J=8.5 Hz, 1H); 4.82 (s, 1H); 4.08 (m, 1H); 3.98 (m, 2H); 3.00 (s, 2H); 2.68-2.38 (m, 6H); 1.80 (m, 4H).

APCI-MS: m/z 446 [MH⁺].

### Example 21

### (2S)-1-(5-Chloro-1′H,3H-spiro[1-benzofuran-2,4′-piperidin]-1′-yl)-3-[(2-methyl-1,3-benzothiazol-4-yl)oxy] propan-2-ol

Step I:

### N-(2-Methoxyphenyl)ethanethioamide

A suspension of N-(2-methoxyphenyl)acetamide (2.4 g, 15 mmol) and P₂S₅ (6.66 g, 15 mmol) in ethyl acetate (60 mL) was refluxed for 2 h, cooled to room temperature and partioned between aqueous NaHCO₃ and CH₂Cl₂. The layers were separated, the organic layer was washed with aqueous NaHCO₃ and H₂O successively, dried over Na₂SO₄, filtered and concentrated in vacuo. The residue was purified by silica gel flash chromatography (0-20% ethyl acetate in petroleum spirit 40-60) to give sub title compound (1.16 g).

¹H-NMR (400 MHz, CDCl₃): δ 9.70 (br.s, 1H); 9.00 (dd, J=1.2, 8.1 Hz, 1H); 7.18 (m, 1H); 7.05-6.94 8 m, 2H); 3.94 (s, 3H); 2.79 (s, 3H).

APCI-MS: m/z 182 [MH⁺].

Step II:

### 4Methoxy-2-methyl-1,3-benzothiazole

An solution of aqueous NaOH solution (4% wt, 25 mL) was added slowly to N-(2-methoxyphenyl)ethanethioamide, followed by a solution of potassium ferricyanide (4.36 g, 13.24 mmol) in water (19 mL). After the addition was completed, the reaction mixture was stirred at room temperature over night, and then extracted with diethyl ether (3×30 mL). The combined organic layer was washed with water (3×10 mL), dried over Na₂SO₄, filtered and concentrated. The residue was purified by silica gel flash chromatography (0-0.8% methanol in dichloromethane containing 0.2% ammonia) to give sub title compound (315 mg).

¹H-NMR (400 MHz, DMSO-d₆): δ 7.41 (dd, J=0.9, 8.1 Hz, 1H); 7.30 (t, J=8.0 Hz, 1H); 6.89 (d, J=8.0 Hz, 1H); 4.01 (s, 3H); 2.83 (s, 3H).

APCI-MS: m/z 180 [MH⁺].

Step III:

### 2-Methyl-1,3-benzothiazol-4-ol

To a cold (ice-water bath) solution of 4-methoxy-2-methyl-1,3-benzothiazole (310 mg, 1.73 mmol) in dichloromethane (8 mL) was slowly added a solution of BBr₃ in dichloromethane (1 M, 4.32 mL, 4.32 mmol). After the addition was completed, the reaction mixture was stirred at room temperature for 24 h. Then the reaction mixture was cooled in ice-water bath, and quenched with methanol (2 mL). The ice bath was removed, the reaction mixture was stirred 20 min. The volatiles were removed in vacuo, and the residue was dissolved in ethyl acetate (200 mL), washed successively with aqueous NaHCO₃ and water. The organic layer was dried over Na₂SO₄, filtered and concentrated. The residue was purified by silica gel flash chromatography (0-0.9% methanol in dichloromethane containing 0.2% ammonia) to give sub title compound (140 mg).

¹H-NMR (400 MHz, DMSO-d₆): δ 10.01 (br.s, 1H); 7.38 (d, J=7.8 Hz, 1H); 7.18 (t, J=8.0 Hz, 1H); 6.82 (d, J=7.8 Hz, 1H); 2.81 (s, 3H).

Step IV:

### 2-Methyl-4-[(2S)-oxiran-2-ylmethoxy]1,3-benzothiazole

A mixture of 2-methyl-1,3-benzothiazol-4-ol (100 mg, 0.6 mmol), (2S)-oxiran-2-ylmethyl-3-nitrobenzenesulfonate (156 mg, 0.6 mmol) and Cs₂CO₃ (254 mg, 0.78 mmol) in DMF (5 mL) was stirred at room temperature over night. The mixture was partitioned between ethyl acetate and water. The organic layer was dried over Na₂SO₄, filtered and concentrated in vacuo. The residue was purified by silica gel flash chromatography (0-40% ethyl acetate in petroleum spirit 40-60) to give sub title compound (95 mg).

¹H-NMR (400 MHz, CDCl₃): δ 7.44 (d, J=8.0 Hz, 1H); 7.28 (t, J=8.0 Hz, 1H); 6.98 (d, J=8.0 Hz, 1H); 4.45 (dd, J=3.7, 11.5 Hz, 1H); 4.30 (dd, J=5.5, 11.5 Hz, 1H); 3.56 (m, 1H); 2.95 (t, J=4.5 Hz, 1H); 2.86 (s, 3H); 2.80 (dd, J=2.6, 4.9 Hz, 1H).

Step V:

### (2S)-1-(5Chloro-1′H,3H-spiro[1-benzofuran-2,4′-piperidin]-1′-yl)-3-[(2-methyl-1,3-benzothiazol-4-yl)oxy]propan-2-ol

A mixture of 5-chloro-3H-spiro[1-benzofuran-2,4′-piperidine] (37 mg, 0.167 mmol) and 2-methyl-4-[(2S)-oxiran-2-ylmethoxy]1,3-benzothiazole (35 mg, 0.167 mmol) in ethanol (1.5 mL) was stirred at 78° C. for 4 h, cooled to room temperature, and the volatiles were removed in vacuo. The residue was purified by silica gel flash chromatography (0-1% methanol in dichloromethane containing 0.2% ammonia) to give title compound (42 mg).

¹H-NMR (400 MHz, CD₃OD): δ 7.49 (d, J=8.0 Hz, 1H); 7.34 (t, J=8.0 Hz, 1H); 7.14 (s, 1H); 7.04 (m, 2H); 6.66 (d, J=8.5 Hz, 1H); 4.32-4.23 (m, 2H); 4.06 (dd, J=6.5, 9.3 Hz, 1H); 3.01 (s, 2H); 2.84 (s, 3H); 2.79-2.64 (m, 6H); 1.99-1.81 (m, 4H).

APCI-MS: m/z 445 [MH⁺].

### Example 22

### (2S)-1-(5-chloro-1′H,3H-spiro[1-benzofuran-2,4′-piperidin]-1′-yl)-3-[(2-methyl-1-benzofuran-4-yl)oxy]propan-2-ol

Step I:

### 2-methyl-1-benzofuran-4-ol

Prepared as described (T. Reichstein, R. Hirt, Helv. Chim. Acta 1933, 16, 121-125).

¹H-NMR (400 MHz, CDCl₃): δ 7.09-7.03 (m, 2H), 6.61 (dd, J=6.6, 2.0 Hz, 1H), 6.45 (s, 1H), 5.56 (br. s, 1H), 2.44 (s, 3H)

Step II:

### 2-methyl-4-[(2S)-oxiran-2-ylmethoxy]-1-benzofuran

A mixture of 2-methyl-1-benzofuran-4-ol (66 mg, 0.45 mmol), (2S)-oxiran-2-ylmethyl-3-nitrobenzenesulfonate (115 mg, 0.45 mmol) and Cs₂CO₃ (176 mg, 0.54 mmol) in DMF (3 mL) was stirred at room temperature over night. The mixture was partitioned between ethyl acetate and water. The organic layer was dried over Na₂SO₄, filtered and concentrated in vacuo. The residue was purified by silica gel flash chromatography (ethyl acetate/n-heptane, 1:1) to give subtitle compound (72 mg, 78%).

¹H-NMR (400 MHz, CDCl₃): δ 7.15-7.07 (m, 2H), 6.63 (d, J=7.4 Hz, 1H), 6.51 (s, 1H), 4.34 (dd, J=11.1, 3.1 Hz, 1H), 4.08 (dd, J=11.1, 5.6 Hz, 1H), 3.42 (m, 1H), 2.93 (t, J=4.5 Hz, 1H), 2.79 (dd, J=4.9, 2.6 Hz, 1H), 2.45 (s, 3H)

Step III:

### (2S)-1-(5-chloro-1′H,3H-spiro[1-benzofuran-2,4′-piperidin]-1′-yl)-3-[(2-methyl-1-benzofuran-4-yl)oxy]propan-2-ol

A mixture of 5-chloro-3H-spiro[1-benzofuran-2,4′-piperidine] (37 mg, 0.17 mmol) and 2-methyl-4-[(2S)-oxiran-2-ylmethoxy]-1-benzofuran (34 mg, 0.17 mmol) in ethanol (2 mL) was stirred at 78° C. overnight, cooled to room temperature, and the volatiles were removed in vacuo. Purification was performed on by semi-preparative HPLC, with acetonitrile/water containing 0.1% trifluoroacetic acid as mobile phase to give the title compound (54 mg, 59%).

¹H-NMR (400 MHz, CDCl₃): δ 7.18-7.07 (m, 4H), 6.69 (d, J=8.5 Hz, 1H), 6.63 (d, J=7.3 Hz, 1H), 6.47 (s, 1H), 4.60 (m, 1H), 4.27 (dd, J=9.6, 4.3 Hz, 1H), 4.09 (dd, J=9.4, 7.6 Hz, 1H), 3.74 (m, 2H), 3.44-3.25 (m, 4H), 3.10 (s, 2H, 2.46 (s, 3H), 2.41 (q, J=17.1 Hz, 2H), 2.15 (t, J=13.5 Hz, 2H)

APCI-MS: m/z 428 [MH⁺].

### Example 23

### 3-{[(2S)-3-(5-chloro-1′H,3H-spiro[1-benzofuran-2,4′-piperidin]-1′-yl)-2-hydroxypropyl]oxy}isonicotinic acid

Step I:

### Ethyl 3-hydroxyisonicotinate

Method A: 3-Hydroxyisonicotinic acid (974 mg, 7.0 mmol) and 1,1′-carbonylbis-1H-imidazole (CDI) (1.3 g, 8 mmol) were stirred in THF (10 mL) at 70° C. 1 h. The mixture was dissolved in a solution of sodium ethylate (0.5 g, 7 mmol) in ethanol (100 mL). After evaporation in vacuo and extraction from DCM and 1M sodium hydrogen carbonate solution the subtitled compound was isolated from the washed organic phase as a yellow solid (803 mg, 69%).

Method B: To a slurry of 3-hydroxyisonicotinic acid (0.56 g, 4 mmol) in ethanol (25 mL) was added thionylchloride (2.35 mL, 32 mmol) at 0° C. The mixture was heated with reflux for 15 h. The solvent was evaporated in vacuo and the residue partitioned between DCM and 1M sodium hydrogencarbonate solution. The subtitle compound was isolated from the washed organic phase as a yellow solid (0.63 g, 94%)

¹H NMR (400 MHz, CDCl₃): δ 10.37 (br. s, 1H), 8.49 (s, 1H), 8.22 (d, J=5.2 Hz, 1H), 7.64 (d, J=5.2 Hz, 1H), 4.46 (q, J=7.2 Hz, 2H), 1.44 (t, J=7.2 Hz, 3H)

APCI-MS: m/z 168 [MH⁺]

Step II:

### Ethyl 3-[(2S)-oxiran-2-ylmethoxy]isonicotinate

To ethyl 3-hydroxyisonicotinate (772 mg, 4.6 mmol) and (2S)-oxiran-2-ylmethyl-3-nitrobenzenesulfonate (1.2 g, 4.6 mmol) dissolved in 1-methyl-2-pyrrolidinone (NMP) (9 mL) was added cesium carbonate (1.6 g, 5 mmol). The mixture was stirred under N₂ at ambient temperature 15 h. After extraction from water and ethylacetate, washing, drying and concentrating the organic phase the subtitled product was obtained as a black oil (0.66 g, 64%).

¹H NMR (400 MHz, CDCl₃) δ 8.45 (s, 1H), 8.35 (d, J=4.9 Hz, 1H), 7.61 (d, J=4.9 Hz, 1H), 4.45 (dd, J=2.8 Hz, J=11.0 Hz, 1H), 4.38 (q, J=7.2 Hz, 2H), 4.15 (dd, J=5.3 Hz, J=11.0 Hz, 1H), 3.42-3.35 (m, 1H), 2.91 (dd, J=4.1 Hz, 5.0 Hz, 1H), 2.86 (dd, J=2.7 Hz, J=5.0 Hz, 1H), 1.39 (t, J=7.2 Hz, 3H)

APCI-MS: m/z 224 [MH⁺]

Step III:

### 3-{[(2S)-3-(5-chloro-1′H,3H-spiro[1-benzofuran-2,4′-piperidin]-1′-yl)-2-hydroxypropyl]oxy}isonicotinic acid

5-Chloro-3H-spiro[1-benzofuran-2,4′-piperidine] (653 mg, 2.9 mnol) and ethyl 3-[(2S)-oxiran-2-ylmethoxy]isonicotinate (650 mg, 2.9 mmol) were dissolved in ethanol (6 mL) and stirred at 80° C. for 15 h. Acetic anhydride (0.3 mL, 3 mmol) was added to trap any unreacted 5-chloro-3H-spiro[1-benzofuran-2,4′-piperidine] as amide. The pH was adjusted to 10 by addition of potassium hydroxide solution (2.5 M) and the mixture was stirred for 3 h at ambient temperature. TFA was added till pH≦2 and the solvent removed in vacuo. The crude product obtained was purified by preparative HPLC using water and acetonitrile containing 0.1% TFA as mobile phase. The title product was obtained as a yellow amorphous solid (bis(trifluoroacetate) salt, 1.15 g, 61%).

¹H NMR (400 MHz, CD₃OD): δ 8.60 (s, 1H), 8.40 (d, J=5.1 Hz, 1H), 7.85 (d, J=5.1 Hz, 1H), 7.21 (s, 1H), 7.12 (d, J=8.5 Hz, 1H), 6.75 (d, J =8.5 Hz, 1H), 4.54-4.45 (m, 1H), 4.41-4.34 (m, 1H), 4.33-4.26 (m, 1H), 3.82-3.63 (m, 1H), 3.58-3.37 (m, 4H), 3.14 (s, 2H), 2.31-2.14 (m, 4H).

APCI-MS: m/z 419 [MH⁺]

### Example 24

### 3-{[(2S)-3-(5-Chloro-1′H,3H-spiro[1-benzofuran-2,4′-piperidin]-1′-yl)-2-hydroxypropyl]oxy}-N-methylisonicotinamide

To a stirred solution of 3-{[(2S)-3-(5-chloro-1′H,3H-spiro[1-benzofuran-2,4′-piperidin]-1′-yl)-2-hydroxypropyl]oxy}isonicotinic acid bis(trifluoroacetate) salt (see Example 23) (38 mg, 91 μmol) and TEA (35 μL, 230 μmol) in THF (4 mL) was added ethyl chloridocarbonate (20 μL, 230 μmol), and the mixture was stirred at ambient temperature for 30 min. Methylamine (2M solution in THF, 125 μL, 230 μmol) was added and the mixture stirred for 1 h. The solvent was evaporated in vacuo and the residue dissolved in sodium methoxide solution (1M in methanol, 3 mL) and stirred at ambient temperature for 30 min. Excess of sodium methoxide was neutralized and the crude product was purified by RP HPLC on silica using acetonitrile and water containing 2 mL 25% ammonia per litre as mobile phase. The title compound was obtained as an amorphous solid (16 mg, 40%).

¹H NMR (300 MHz, acetone-d₆) δ 8.58 (s, 1H), 8.45.-8.30 (br. s, 1H), 8.34 (d, J=4.7 Hz, 1H), 7.83 (d, J=4.9 Hz, 1H), 7.19-7.16 (m, 1H), 7.09 (d, J=8.5 Hz, 1H), 6.70 (d, J=8.5 Hz, 1H), 4.57-4.47 (m, 1H), 4.33-4.20 (m, 2H), 3.05 (s, 2H), 2.91 (d, J=4.7 Hz, 3H), 2.87-2.57 (m, 6H), 2.00-1.79 (m, 4H)

APCI-MS: m/z 432 [MH⁺]

### Example 25

### (3S)-1-(3-{[(2S)-3-(5-Chloro-1′H,3H-spiro[1-benzofuran-2,4′-piperidin]-1′-yl)-2-hydroxypropyl]oxy}isonicotinoyl)pyrrolidin-3-ol

A solution of 3-{[(2S)-3-(5-chloro-1′H,3H-spiro[1-benzofiuran-2,4′-piperidin]-1′-yl)-2-hydroxypropyl]oxy}isonicotinic acid bis(trifluoroacetate) salt (see Example 23) (65 mg, 0.1 mmol) in THF (3 mL) and 1,1′-carbonylbis-1H-imidazole (CDI) (36 mg, 0.22 mmol) was stirred at 70° C. for 30 min. (3S)-Pyrrolidin-3-ol (30 mg, 0.33 mmol) was added and the mixture stirred for 2 h at 70° C. The solvent was evaporated in vacuo and the residue partitioned between ethylacetate and water (pH≈10). The crude product obtained from the washed organic phase was purified by RP HPLC using acetonitrile and water containing 0.1% TFA as mobile phase. The title product was obtained as a white amorphous solid (31 mg, 40%).

¹H NMR (400 MHz, CD₃OD): δ 8.66 (s, 0.51), 8.64 (s, 0.5H), 8.474 (d, J=5.2 Hz, 0.5H), 8.469 (d, J=5.2 Hz, 0.5H), 7.66 (d, J=5.5 Hz, 0.5H), 7.65 (d, J=5.5 Hz, 0.5H), 7.20 (s, 1H), 7.11 (d, J=8.5 Hz, 1H), 6.74 (d, J=8.5 Hz, 1H), 4.57-4.52 (m, 0.5H), 4.52-4.45 (m, 1H), 4.45-4.41 (m, 0.5H), 4.37-4.28 (m, 2H), 3.84-3.33 (m, 9H), 3.20 (d, J=11.2 Hz, 1H), 3.13 (s, 2H), 2.27-1.93 (m, 6H) (mixture of atropisomers, 1:1 ratio).

APCI-MS: m/z 488 [MH⁺]

THP-1 Chemotaxis Assay

Introduction

The assay measures the chemotactic response elicited by MIP-1α chemokine in the human monocytic cell line THP-1. Compounds are evaluated by their ability to depress the chemotactic response to a standard concentration of MIP-1α chemokine.

Methods

Culture of THP-1 Cells

Cells are thawed rapidly at 37° C. from frozen aliquots and resuspended in a 25 cm flask containing 5 ml of RPMI-1640 medium supplemented with Glutamax and 10% heat inactivated fetal calf serum without antibiotics (RPMI+10% HIFCS). At day 3 the medium is discarded and replaced with fresh medium.

THP-1 cells are routinely cultured in RPMI-1640 medium supplemented with 10% heat inactivated fetal calf serum and glutamax but without antibiotics. Optimal growth of the cells requires that they are passaged every 3 days and that the minimum subculture density is 4×10⁵ cells/ml.

Chemotaxis Assay

Cells are removed from the flask and washed by centrifugation in RPMI+10% HIFCS+glutamax. The cells are then resuspended at 2×10⁷ cells/ml in fresh medium (RPMI+10% HIFCS+glutamax) to which is added calcein-AM (5 μl of stock solution to 1 ml to give a final concentration of 5×10⁻⁶M). After gentle mixing the cells are incubated at 37° C. in a CO₂ incubator for 30 minutes. The cells are then diluted to 50 ml with medium and washed twice by centrifugation at 400×g. Labelled cells are then resuspended at a cell concentration of 1×10⁷ cells/ml and incubated with an equal volume of MIP-1α antagonist (10⁻¹⁰M to 10⁻⁶M final concentration) for 30 minutes at 37° C. in a humidified CO₂ incubator.

Chemotaxis is performed using Neuroprobe 96-well chemotaxis plates employing 8 μm filters (cat no. 101-8). Thirty microlitres of chemoattractant supplemented with various concentrations of antagonists or vehicle are added to the lower wells of the plate in triplicate. The filter is then carefully positioned on top and then 25 μl of cells preincubated with the corresponding concentration of antagonist or vehicle is added to the surface of the filter. The plate is then incubated for 2 hours at 37° C. in a humidified CO₂ incubator. The cells remaining on the surface are then removed by adsorption and the whole plate is centrifuged at 2000 rpm for 10 minutes. The filter is then removed and the cells that have migrated to the lower wells are quantified by the fluorescence of cell associated calcein-AM. Cell migration is then expressed in fluorescence units after subtraction of the reagent blank and values are standardized to % migration by comparing the fluorescence values with that of a known number of labelled cells. The effect of antagonists is calculated as % inhibition when the number of migrated cells is compared with vehicle.

1. A compound of formula 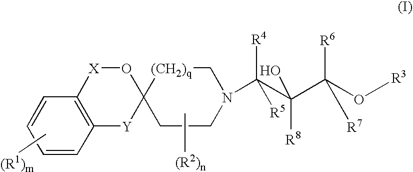
wherein
m is 0, 1, 2, 3 or 4;
each R¹ independently represents halogen, cyano, hydroxyl, C₁-C₆ alkyl, C₁-C₆ haloalkyl, C₁-C₆ alkoxy, C₁-C₆ alkylsulphonyl or sulphonamido;
X represents a bond or —CH₂— and Y represents a bond or —CH₂—, provided that X and Y do not both simultaneously represent a bond or —CH₂—;
n is 0, 1 or 2;
each R² independently represents halogen, C₁-C₆ alkyl or C₁-C₆ haloalkyl;
q is 0 or 1;
R³ represents a saturated or unsaturated 5- to 10-membered ring system other than phenyl, which ring system may comprise at least one ring heteroatom selected from nitrogen, oxygen and sulphur, the ring system being optionally substituted with at least one substituent selected from halogen, cyano, oxo, nitro, hydroxyl, carboxyl, —C(O)H, —NR⁹R¹⁰, —C(O)NR¹¹R¹², —NHC(O)R¹³, —NHSO₂R¹⁴, —SO₂NR¹⁵R¹⁶, —NHC(O)NR¹⁷R¹⁸, C₁-C₆ alkyl, C₁-C₆ alkoxy, C₁-C₆ alkylthio, C₁-C₆ alkylsulphonyl, C₁-C₆ haloalkyl, C₁-C₆ alkoxyC₁-C₆ alkyl, C₁-C₆ alkylcarbonyl, phenylcarbonyl, C₃-C₆ cycloalkyl, C₃-C₆ cycloalkylmethyl and a saturated or unsaturated 5- to 6-membered heterocyclic ring comprising at least one ring heteroatom selected from nitrogen, oxygen and sulphur;
R⁴, R⁵, R⁶, R⁷ and R⁸ each independently represent hydrogen, halogen, C₁-C₆ alkyl or C₁-C₆ haloalkyl;
R⁹ and R¹⁰ each independently represent hydrogen, C₁-C₆ alkyl or C₃-C₆ cycloalkyl;
R¹¹ and R¹² each independently represent hydrogen, C₁-C₆ alkyl or C₃-C₆ cycloalkyl, or R¹¹ and R¹² together with the nitrogen atom to which they are attached form a 4- to 7-membered saturated heterocyclic ring which may be optionally substituted with at least one substituent selected from hydroxyl;
R¹³ and R¹⁴ each independently represent C₁-C₆ alkyl, C₃-C₆ cycloalkyl or C₁-C₄ haloalkyl;
R¹⁵ and R¹⁶ each independently represent hydrogen, C₁-C₆ alkyl or C₃-C₆ cycloalkyl, or R¹⁵ and R¹⁶ together with the nitrogen atom to which they are attached form a 4- to 7-membered saturated heterocyclic ring which may be optionally substituted with at least one substituent selected from hydroxyl; and
R¹⁷ and R¹⁸ each independently represent hydrogen, C₁-C₆ alkyl or C₃-C₆ cycloalkyl, or R¹⁷ and R¹⁸ together with the nitrogen atom to which they are attached form a 4- to 7-membered saturated heterocyclic ring which may be optionally substituted with at least one substituent selected from hydroxyl;
or a pharmaceutically acceptable salt or solvate thereof.

2. A compound according to claim 1, wherein X represents a bond and Y represents —CH₂—.

3. A compound according to claim 1, wherein q is 1.

4. A compound according to claim 1, wherein m is 1 and R¹ represents halogen.

5. A compound according to claim 1, wherein R³ represents an unsaturated 6- to 10-membered ring system other than phenyl, which ring system may comprise one or two ring heteroatoms independently selected from nitrogen and oxygen, or two ring heteroatoms consisting of nitrogen and sulphur, the ring system being optionally substituted with one, two or three substituents independently selected from halogen, oxo, nitro, —NH₂, C₁-C₄ alkyl, C₁-C₄ alkoxy, C₁-C₄ alkylthio, C₁-C₄ haloalkyl, C₁-C₄ alkoxyC₁-C₄ alkyl, C₁-C₄ alkylcarbonyl, C₃-C₆ cycloalkylmethyl, —C(O)NR¹¹R¹², carboxyl, and a saturated or unsaturated 5- to 6-membered heterocyclic ring comprising one or two ring heteroatoms independently selected from nitrogen and sulphur.

6. A compound according to claim 5, wherein the unsaturated 6- to 10-membered ring system is selected from quinolinyl, 1,2-dihydroquinolinyl, 1,2,3,4-tetrahydroquinolinyl, 2,3-dihydrobenzoxazinyl, 1,2,3,4-tetrahydroquinazolinyl, naphthyl, pyridinyl, benzofuranyl, benzothiazolyl, pyrimidinyl, isoquinolinyl and quinazolinyl.

7. A compound according to claim 1, wherein R⁴, R⁵, R⁶, R⁷ and R⁸ each independently represent hydrogen.

8. A compound according to claim 1 selected from:
8-{[(2S)-3-(5-Chloro-1′H,3H-spiro[1-benzofuran-2,4′-piperidin]-1′-yl)-2-hydroxypropyl]oxy}-3,4-dihydroquinolin-2(1H)-one,
8-{[(2S)-3-(5-Chloro-1′H,3H-spiro[1-benzofuran-2,4′-piperidin]-1′-yl)-2-hydroxypropyl]oxy}quinolin-2(1H)-one,
5-{[(2S)-3-(5-Chloro-1′H,3H-spiro[1-benzofuran-2,4′-piperidin]-1′-yl)-2-hydroxypropyl]oxy}-2H-1,4-benzoxazin-3(4H)-one,
8-{[(2S)-3-(5-Chloro-1′H,3H-spiro[1-benzofuran-2,4′-piperidin]-1′-yl)-2-hydroxypropyl]oxy}quinazoline-2,4(1H,3H)-dione trifluoroacetate (salt),
(2S)-1-(5-Chloro-1′H,3H-spiro[1-benzofuran-2,4′-piperidin]-1′-yl)-3-(1-naphthyloxy)propan-2-ol trifluoroacetate (salt),
(2S)-1-(5-Chloro-1′H,3H-spiro[1-benzofuran-2,4′-piperidin]-1′-yl)-3-[(6-methyl-2-nitropyridin-3-yl)oxy]propan-2-ol trifluoroacetate (salt),
1-(6-{[(2S)-3-(5-Chloro-1′H,3H-spiro[1-benzofuran-2,4′-piperidin]-1′-yl)-2-hydroxypropyl]oxy}-4,7-dimethoxy-1-benzofuran-5-yl)ethanone trifluoroacetate (salt),
(2S)-1-[(6-Chloropyridin-2-yl)oxy]-3-(5-chloro-1′H,3H-spiro[1-benzofuran-2,4′-piperidin]-1′-yl)propan-2-ol trifluoroacetate (salt),
(2S)-1-(5-Chloro-1′H,3H-spiro[1-benzofuran-2,4′-piperidin]-1′-yl)-3-{[7-(trifluoromethyl)quinolin-4-yl]oxy}propan-2-ol trifluoroacetate (salt),
(2S)-1-(5-Chloro-1′H,3H-spiro[1-benzofuran-2,4′-piperidin]-1′-yl)-3-[(2-iodo-6-methylpyridin-3-yl)oxy]propan-2-ol trifluoroacetate (salt),
(2S)-1-(5-Chloro-1′H,3H-spiro[1-benzofuran-2,4′-piperidin]-1′-yl)-3-{[5-(cyclopropylmethyl)-6-methyl-2-pyridin-4-ylpyrimidin-4-yl]oxy}propan-2-ol trifluoroacetate (salt),
(2S)-1-(5-Chloro-1′H,3H-spiro[1-benzofuran-2,4′-piperidin]-1′-yl)-3-(quinolin-8-yloxy)propan-2-ol trifluoroacetate (salt),
(2S)-1-(5-Chloro-1′H,3H-spiro[1-benzofuran-2,4′-piperidin]-1′-yl)-3-(isoquinolin-5-yloxy)propan-2-ol trifluoroacetate (salt),
(2S)-1-[(6-Bromoquinazolin-4-yl)oxy]-3-(5-chloro-1′H,3H-spiro[1-benzofuran-2,4′-piperidin]-1′-yl)propan-2-ol trifluoroacetate (salt),
(2S)-1-(5-Chloro-1′H,3H-spiro[1-benzofuran-2,4′-piperidin]-1′-yl)-3-{[2-(2-thienyl)-6-(trifluoromethyl)pyrimidin-4-yl]oxy}propan-2-ol trifluoroacetate (salt),
(2S)-1-(5-Chloro-1′H,3H-spiro[1-benzofuran-2,4′-piperidin]-1′-yl)-3-(quinolin-5-yloxy)propan-2-ol trifluoroacetate (salt),
(2S)-1-(5-Chloro-1′H,3H-spiro[1-benzofuran-2,4′-piperidin]-1′-yl)-3-[(2,3,4-trichloro-1-naphthyl)oxy]propan-2-ol trifluoroacetate (salt),
(2S)-1-(5-Chloro-1′H,3H-spiro[1-benzofuran-2,4′-piperidin]-1′-yl)-3-{[1-(1,3-dithiolan-2-yl)-2-naphthyl]oxy}propan-2-ol trifluoroacetate (salt),
(2S)-1-{[5-Butyl-6-(methoxymethyl)-2-(methylthio)pyrimidin-4-yl]oxy}-3-(5-chloro-1′H,3H-spiro[1-benzofuran-2,4′-piperidin]-1′-yl)propan-2-ol trifluoroacetate (salt),
(2S)-1-[(2-Amino-1,3-benzothiazol-4-yl)oxy]-3-(5-chloro-1′H,3H-spiro[1-benzofuran-2,4′-piperidin]-1′-yl)propan-2-ol,
(2S)-1-(5-Chloro-1′H,3H-spiro[1-benzofuran-2,4′-piperidin]-1′-yl)-3-[(2-methyl-1,3-benzothiazol-4-yl)oxy]propan-2-ol,
(2S)-1-(5-chloro-1′H,3H-spiro[1-benzofuran-2,4′-piperidin]-1′-yl)-3-[(2-methyl-1-benzofuran-4-yl)oxy]propan-2-ol,
3-{[(2S)-3-(5-chloro-1′H,3H-spiro[1-benzofuran-2,4′-piperidin]-1′-yl)-2-hydroxypropyl]oxy}isonicotinic acid,
3-{[(2S)-3-(5-Chloro-1′H,3H-spiro[1-benzofuran-2,4′-piperidin]-1′-yl)-2-hydroxypropyl]oxy}-N-methylisonicotinamide,
(3S)-1-(3-{[(2S)-3-(5-Chloro-1′H,3H-spiro[1-benzofuran-2,4′-piperidin]-1′-yl)-2-hydroxypropyl]oxy}isonicotinoyl)pyrrolidin-3-ol,
and pharmaceutically acceptable salts and solvates of any one thereof.

9. A process for the preparation of a compound of formula (I) or a pharmaceutically acceptable salt or solvate thereof as defined in claim 1 which comprises,
(a) reacting a compound of formula 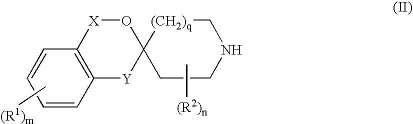
wherein m, R¹, n, R², q, X and Y are as defined in formula (I), with a compound of formula 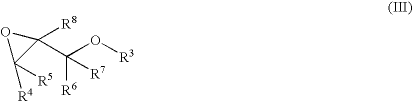
wherein R³, R⁴, R⁵, R⁶, R⁷ and R⁸ are as defined in formula (I); or
(b) reacting a compound of formula 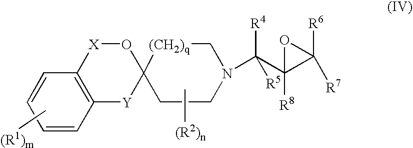
wherein m, R¹, n, R², q, X, Y, R⁴, R⁵, R⁶, R⁷ and R⁸ are as defined in formula (I), with a compound of formula
HO—R³ (V)
wherein R³ is as defined in formula (I), in the presence of a suitable base;
(c) when R³ is substituted with —C(O)NR¹¹R¹², reacting a compound of formula 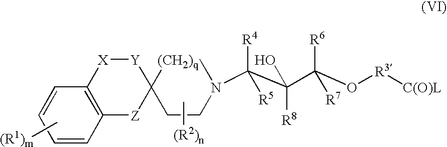
wherein L represents a leaving group (e.g. a hydroxyl group), R^{3′} is a saturated or unsaturated 5- to 10-membered ring system other than phenyl, which ring system may comprise at least one ring heteroatom selected from nitrogen, oxygen and sulphur, and m, R¹, n, R², q, X, Y, Z, R⁴, R⁵, R⁶, R⁷ and R⁸ are as defined in formula (I), with a compound of formula (VII),
NHR¹¹R¹² (VII)
wherein R¹¹ and R¹² are as defined in formula (I), in the presence of a suitable coupling reagent;
and optionally after (a), (b) or (c) forming a pharmaceutically acceptable salt or solvate.

10. A pharmaceutical composition comprising a compound of formula (I) or a pharmaceutically acceptable salt or solvate thereof as claimed in claim 1 in association with a pharmaceutically acceptable adjuvant, diluent or carrier.

11. A process for the preparation of a pharmaceutical composition comprising a compound of formula (I) or a pharmaceutically acceptable salt or solvate thereof as claimed in claim 1 in association with a pharmaceutically acceptable adjuvant, diluent or carrier, which comprises mixing a compound of formula (I) or a pharmaceutically acceptable salt or solvate thereof as claimed in claim 1 with a pharmaceutically acceptable adjuvant, diluent or carrier.

12. (canceled)

13. A method of treating a disease or condition in which modulation of chemokine receptor activity is beneficial, the method comprising administering to a patient in need thereof a compound of formula (I) or a pharmaceutically acceptable salt or solvate thereof as claimed in claim 1.

14. A method of treating rheumatoid arthritis, the method comprising administering to a patient in need thereof a compound of formula (I) or a pharmaceutically acceptable salt or solvate thereof as claimed in claim 1.

15. A method of treating chronic obstructive pulmonary disease, the method comprising administering to a patient in need thereof a compound of formula (I) or a pharmaceutically acceptable salt or solvate thereof as claimed in claim 1.

16. A method of treating asthma, the method comprising administering to a patient in need thereof a compound of formula (I) or a pharmaceutically acceptable salt or solvate thereof as claimed in claim 1.

17. A method of treating multiple sclerosis, the method comprising administering to a patient in need thereof a compound of formula (I) or a pharmaceutically acceptable salt or solvate thereof as claimed in claim 1.

18. A method of treating an inflammatory disease which comprises administering to a patient in need thereof a therapeutically effective amount of a compound of formula (I) or a pharmaceutically acceptable salt or solvate thereof as claimed in claims 1.

19. A method of treating an airways disease which comprises administering to a patient in need thereof a therapeutically effective amount of a compound of formula (I) or a pharmaceutically acceptable salt or solvate thereof as claimed in claim 1.
